(19) United States
(12) Patent Application Publication
(10) Pub. No.: US 2023/0270723 A1
(43) Pub. Date: Aug. 31, 2023

(54) GLUCOKINASE ACTIVATOR FOR TREATING DIABETES WITH HEPATIC IMPAIRMENT

(71) Applicant: HUA Medicine (Shanghai) Ltd., Shanghai (CN)

(72) Inventors: Li CHEN, Shanghai (CN); Shuang REN, Shanghai (CN); Chengfeng JIAO, Shanghai (CN)

(21) Appl. No.: 18/008,005

(22) PCT Filed: Jun. 3, 2021
(86) PCT No.: PCT/CN2021/098107

§ 371 Date: Dec. 2, 2022

(30) Foreign Application Priority Data
Jun. 4, 2020 (WO) PCT/CN2020/094372

(51) Int. Cl. A61K 31/4155; A61K 45/06; A61K 9/00; A61P 3/10

(52) U.S. Cl. CPC A61K 31/4155; A61K 45/06; A61K 9/0053; A61P 3/10

(57) ABSTRACT

Provided herein is a method of treating, preventing, or ameliorating one or more symptoms of a glucokinase-mediated disorder, disease, or condition in a hepatically impaired subject with a glucokinase activator (GKA), for example, dorzagliatin. Also provided herein is a method of treating, preventing, or ameliorating one or more symptoms of a diabetes in a hepatically impaired subject with a GKA. Additionally, provided herein is a method of treating, preventing, or ameliorating one or more symptoms of a chronic liver disease with a GKA.

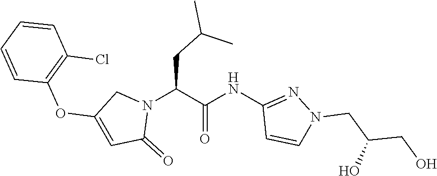 [SMILES: CC(C)C[C@@H](C(=O)Nc1ccn(C[C@@H](O)CO)n1)N1CC(Oc2ccccc2Cl)=CC1=O]

### CROSS-REFERENCE TO RELATED APPLICATIONS

This application claims the priority of International Patent Application No. PCT/CN2020/094372, filed Jun. 4, 2020, the entirety of which is incorporated herein by reference.

### FIELD

Provided herein is a method of treating, preventing, or ameliorating one or more symptoms of a glucokinase-mediated disorder, disease, or condition in a hepatically impaired subject with a glucokinase activator (GKA). Also provided herein is a method of treating, preventing, or ameliorating one or more symptoms of a diabetes in a hepatically impaired subject with a GKA. Additionally, provided herein is a method of treating, preventing, or ameliorating one or more symptoms of a chronic liver disease with a GKA.

### BACKGROUND

Diabetes mellitus is a major health issue in the world. Nearly half a billion people are living with diabetes worldwide in 2019. IDF Diabetes Atlas; 9th ed.; International Diabetes Federation; 2019. Type 2 diabetes (T2DM), a non-insulin dependent diabetes mellitus, accounts for more than 90% of diabetes in the world. Id.; Wu et al., Int. J. Med. Sci. 2014, 11, 1185-1200. Diabetes mellitus is also the leading cause of a liver disease. Tolman et al., Diabetes Care 2007, 30, 734-743; Papazafiropoulou and Melidonis, World J. Met-Anal. 2019, 7, 380-388. T2DM often coexists with a chronic liver disease, such as nonalcoholic fatty liver disease (NAFLD), cirrhosis, hepatocellular carcinoma, and acute liver failure. Tolman et al., Diabetes Care 2007, 30, 734-743. Because the liver is the primary site of drug metabolism, the decline in hepatic function can impair the clearance and metabolism of an antidiabetic agent. Papazafiropoulou and Melidonis, World J. Met-Anal. 2019, 7, 380-388. As a consequence, many antidiabetic agents are not recommended or require dose adjustment for treating hepatically impaired diabetic patients. Id. For example, metformin, generally the first medication prescribed for T2DM, is contraindicated in diabetic patients with clinical or laboratory evidence of a hepatic disease. Id.; GLUCOPHAGE® and GLUCOPHASE® XR Prescription Label (May 2018).

Glucokinase (GK) plays a central role in stabilizing the blood glucose balance in the human body. GK as a glucose sensor in glucose homeostasis, regulates the secretion of glucagon, insulin, and GLP-1 stimulated by glucose. GK is mainly distributed in the liver, where it rapidly converts glucose into hepatic glycogen for storage in response to elevated blood glucose and meanwhile lowers the glucose level in the blood. A defect of glucokinase causes impaired glucose tolerance (IGT) and type 2 diabetes. However, there is currently no GKAs approved for clinical use. Therefore, there is a clinical need to be met in treating a diabetes with hepatic impairment.

### SUMMARY OF THE DISCLOSURE

Provided herein is a method of treating, preventing, or ameliorating one or more symptoms of a glucokinase-mediated disorder, disease, or condition in a subject with hepatic impairment, comprising administering to the subject in need thereof a therapeutically effective amount of a glucokinase activator (GKA).

Also provided herein is a method of treating, preventing, or ameliorating one or more symptoms of a diabetes in a subject with hepatic impairment, comprising administering to the subject in need thereof a therapeutically effective amount of a GKA.

Additionally, provided herein is a method of treating hyperglycemia in a subject with hepatic impairment, comprising administering to the subject in need thereof a therapeutically effective amount of a GKA.

Furthermore, provided herein is a method of treating prediabetes in a subject with hepatic impairment, comprising administering to the subject in need thereof a therapeutically effective amount of a GKA.

Provided herein is a method of treating, preventing, or ameliorating one or more symptoms of a chronic liver disease in a subject, comprising administering to the subject in need thereof a therapeutically effective amount of a GKA.

Provided herein is a method of slowing the progression of a chronic liver disease to end-stage liver disease (i.e., liver failure) in a subject, comprising administering to a subject in need thereof a therapeutically effective amount of a GKA.

Provided herein is a method of modulating the activity of a glucokinase in a subject with hepatic impairment, comprising administering to the subject in need thereof an effective amount of a GKA.

Provided herein is a method of treating, preventing, or ameliorating one or more symptoms of diabetes in a subject with hepatic impairment, comprising administering to the subject in need thereof a therapeutically effective amount of (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof.

Provided herein is a method of treating, preventing, or ameliorating one or more symptoms of a chronic liver disease in a subject, comprising administering to the subject in need thereof a therapeutically effective amount of (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 22nd ed.; Allen Ed.: Philadelphia, Pa., 2012; Handbook of Pharmaceutical Excipients, 8th ed.; Sheskey et al., Eds.; The Pharmaceutical Press: 2017; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I), iodine-129 (129I) and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N) nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C) nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I) iodine-129 (¹²⁹I) and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., 1H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “a tautomer, a mixture of two or more tautomers, or an isotopic variant thereof; or a pharmaceutically acceptable salt, solvate, hydrate, or prodrug thereof” has the same meaning as the phrase “(i) a tautomer, a mixture of two or more tautomers, or an isotopic variant of the compound referenced therein; or (ii) a pharmaceutically acceptable salt, solvate, hydrate, or prodrug of the compound referenced therein, or (iii) a pharmaceutically acceptable salt, solvate, hydrate, or prodrug of a tautomer, a mixture of two or more tautomers, or an isotopic variant of the compound referenced therein.”

### Methods of Treatment

In one embodiment, provided herein is a method of treating, preventing, or ameliorating one or more symptoms of a glucokinase-mediated disorder, disease, or condition in a subject with hepatic impairment, comprising administering to the subject in need thereof a therapeutically effective amount of a glucokinase activator (GKA).

In one embodiment, the glucokinase-mediated disorder, disease, or condition is a diabetes, type 1 diabetes, type 2 diabetes, diabetic nephropathy, hyperglycemia, postprandial hyperglycemia, postabsorptive hyperglycemia, hyperinsulinemia, hyperlipidemia, impaired fasting blood glucose (IFG), impaired glucose tolerance (IGT), insulin resistance syndrome, latent autoimmune diabetes in adults (LADA), metabolic syndrome, obesity, or prediabetes.

In one embodiment, the glucokinase-mediated disorder, disease, or condition is a metabolic disorder. In another embodiment, the glucokinase-mediated disorder, disease, or condition is a diabetes. In yet another embodiment, the glucokinase-mediated disorder, disease, or condition is type-1 diabetes. In yet another embodiment, the glucokinase-mediated disorder, disease, or condition is type-2 diabetes. In yet another embodiment, the glucokinase-mediated disorder, disease, or condition is hyperglycemia. In yet another embodiment, the glucokinase-mediated disorder, disease, or condition is prediabetes. In yet another embodiment, the glucokinase-mediated disorder, disease, or condition is obesity. In yet another embodiment, the glucokinase-mediated disorder, disease, or condition is a liver disease. In still another embodiment, the glucokinase-mediated disorder, disease, or condition is a chronic liver disease.

In another embodiment, provided herein is a method of treating, preventing, or ameliorating one or more symptoms of diabetes in a subject with hepatic impairment, comprising administering to the subject in need thereof a therapeutically effective amount of a GKA.

In one embodiment, the diabetes is type 1 diabetes. In another embodiment, the diabetes is type 2 diabetes.

In one embodiment, the diabetes is an untreated diabetes. In another embodiment, the diabetes is untreated type 1 diabetes. In yet another embodiment, the diabetes is untreated type 2 diabetes.

In one embodiment, the diabetes is a treatment-resistant diabetes. In another embodiment, the diabetes is treatment-resistant type 1 diabetes. In yet another embodiment, the diabetes is treatment-resistant type 2 diabetes.

In certain embodiments, the diabetes is a diabetes with persistent hyperglycemia. In certain embodiments, the diabetes is a diabetes with a glycated hemoglobin level (HbA1c) of no less than about 7%. In certain embodiments, the diabetes is a diabetes with an HbA1c of no less than about 8%. In certain embodiments, the diabetes is a diabetes with an HbA1c of no less than about 9%. In certain embodiments, the diabetes is a diabetes with an HbA1c of no less than about 10%.

In certain embodiments, the diabetes is a diabetes with an HbA1c of no less than about 64 mmol/mol. In certain embodiments, the diabetes is a diabetes with an HbA1c of no less than about 75 mmol/mol. In certain embodiments, the diabetes is a diabetes with an HbA1c of no less than about 86 mmol/mol.

In certain embodiments, the treatment-resistant diabetes is a diabetes with persistent hyperglycemia despite pharmacological treatment with at least three oral glucose-lowering medications. In certain embodiments, the treatment-resistant diabetes is a diabetes with an HbA1c of no less than about 7% despite pharmacological treatment with at least three oral glucose-lowering medications. In certain embodiments, the treatment-resistant diabetes is a diabetes with an HbA1c of no less than about 8% despite pharmacological treatment with at least three oral glucose-lowering medications. In certain embodiments, the treatment-resistant diabetes is a diabetes with an HbA1c of no less than about 9% despite pharmacological treatment with at least three oral glucose-lowering medications. In certain embodiments, the treatment-resistant diabetes is a diabetes with an HbA1c of no less than about 10% despite pharmacological treatment with at least three oral glucose-lowering medications.

In certain embodiments, the treatment-resistant diabetes is a diabetes with an HbA1c of no less than about 64 mmol/mol despite pharmacological treatment with at least three oral glucose-lowering medications. In certain embodiments, the treatment-resistant diabetes is a diabetes with an HbA1c of no less than about 75 mmol/mol despite pharmacological treatment with at least three oral glucose-lowering medications. In certain embodiments, the treatment-resistant diabetes is a diabetes with an HbA1c of no less than about 86 mmol/mol despite pharmacological treatment with at least three oral glucose-lowering medications.

In certain embodiments, the treatment-resistant diabetes is a diabetes with persistent poorly-controlled diabetes despite standard care with three oral glucose-lowering medications.

In one embodiment, the treatment-resistant diabetes is resistant to a dipeptidyl peptidase 4 (DPP-4) inhibitor, a glucagon-like peptide-1 (GLP-1) agonist, an insulin, a meglitinide, metformin, an SGLT2 inhibitor, a sulfonylurea, or a thiazolidinedione, or a combination thereof.

In one embodiment, the treatment-resistant diabetes is resistant to a DPP-4 inhibitor. In another embodiment, the treatment-resistant diabetes is resistant to metformin. In yet another embodiment, the treatment-resistant diabetes is resistant to an SGLT-2 inhibitor. In yet another embodiment, the treatment-resistant diabetes is resistant to a DPP-4 inhibitor and metformin. In yet another embodiment, the treatment-resistant diabetes is resistant to a DPP-4 inhibitor and an SGLT-2 inhibitor. In yet another embodiment, the treatment-resistant diabetes is resistant to an SGLT-2 inhibitor and metformin. In still another embodiment, the treatment-resistant diabetes is resistant to a DPP-4 inhibitor, metformin, and an SGLT-2 inhibitor.

In certain embodiments, the treatment-resistant diabetes is resistant to a DPP-4 inhibitor. In certain embodiments, the treatment-resistant diabetes is resistant to alogliptin, dutogliptin, evogliptin, gemigliptin, gosogliptin, linagliptin, omarigliptin, saxagliptin, sitagliptin, teneligliptin, trelagliptin, or vildagliptin. In certain embodiments, the treatment-resistant diabetes is resistant to alogliptin, evogliptin, gemigliptin, gosogliptin, linagliptin, omarigliptin, saxagliptin, sitagliptin, teneligliptin, trelagliptin, or vildagliptin. In certain embodiments, the treatment-resistant diabetes is resistant to alogliptin, linagliptin, saxagliptin, or sitagliptin.

In certain embodiments, the treatment-resistant diabetes is resistant to a GLP-1 receptor agonist. In certain embodiments, the treatment-resistant diabetes is resistant to albiglutide, dulaglutide, exenatide, liraglutide, lixisenatide, or semaglutide.

In certain embodiments, the treatment-resistant diabetes is resistant to an insulin. In certain embodiments, the treatment-resistant diabetes is resistant to a fast-acting insulin, a short-acting insulin, an intermediate-acting insulin, a long-acting insulin, or an ultra-long acting insulin.

In certain embodiments, the treatment-resistant diabetes is resistant to a meglitinide. In certain embodiments, the treatment-resistant diabetes is resistant to nateglinide or repaglinide.

In certain embodiments, the treatment-resistant diabetes is resistant to an SGLT2 inhibitor. In certain embodiments, the treatment-resistant diabetes is resistant to bexagliflozin, canagliflozin, dapagliflozin, empagliflozin, ertugliflozin, ipragliflozin, luseogliflozin, phlorizin, remogliflozin, serglifozin, sotagliflozin, or tofogliflozin. In certain embodiments, the treatment-resistant diabetes is resistant to canagliflozin, dapagliflozin, empagliflozin, ertugliflozin, ipragliflozin, or tofogliflozin. In certain embodiments, the treatment-resistant diabetes is resistant to canagliflozin, dapagliflozin, empagliflozin, or ertugliflozin.

In certain embodiments, the treatment-resistant diabetes is resistant to a sulfonylurea. In certain embodiments, the treatment-resistant diabetes is resistant to chlorpropamide, gliclazide, glimepiride, or tolazamide.

In certain embodiments, the treatment-resistant diabetes is resistant to a thiazolidinedione. In certain embodiments, the treatment-resistant diabetes is resistant to balaglitazone, ciglitazone, darglitazone, englitazone, lobeglitazone, netoglitazone, pioglitazone, rivoglitazone, rosiglitazone, or troglitazone. In certain embodiments, the treatment-resistant diabetes is resistant to lobeglitazone, rosiglitazone, or pioglitazone.

In certain embodiments, the subject with a treatment-resistant diabetes fails a monotherapy. In certain embodiments, the subject with a treatment-resistant diabetes fails a dual-agent therapy.

In yet another embodiment, provided herein is a method of treating hyperglycemia in a subject with hepatic impairment, comprising administering to the subject in need thereof a therapeutically effective amount of a GKA.

In still another embodiment, provided herein is a method of treating prediabetes in a subject with hepatic impairment, comprising administering to the subject in need thereof a therapeutically effective amount of a GKA.

In certain embodiments, the subject has mild hepatic impairment with a Child-Pugh score from 5 to 6. In certain embodiments, the subject has moderate hepatic impairment with a Child-Pugh score from 7 to 9. In certain embodiments, the subject has severe hepatic impairment with a Child-Pugh score from 10 to 15. In certain embodiments, the subject has end-stage liver disease (ESLD) or liver failure. The Child-Pugh score is a system for assessing the prognosis—including the required strength of treatment—of chronic liver disease.

In certain embodiments, the subject has a liver disease. In certain embodiments, the subject has an acute liver disease. In certain embodiments, the subject has a chronic liver disease.

In one embodiment, provided herein is a method of treating, preventing, or ameliorating one or more symptoms of a chronic liver disease (CLD) in a subject, comprising administering to the subject in need thereof a therapeutically effective amount of a GKA.

In another embodiment, provided herein is a method of slowing the progression of a chronic liver disease to end-stage liver disease (ESLD) in a subject, comprising administering to a subject in need thereof a therapeutically effective amount of a GKA.

In one embodiment, the chronic liver disease is a mild CLD with a Child-Pugh score from 5 to 6. In another embodiment, the chronic liver disease is a moderate CLD with a Child-Pugh score from 7 to 9. In yet another embodiment, the chronic liver disease is a severe CLD with a Child-Pugh score from 10 to 15. In yet another embodiment, the chronic liver disease is ESLD or liver failure.

In one embodiment, the chronic liver disease is diabetic liver disease (DLD). In certain embodiments, the diabetic liver disease is type-1 DLD. In certain embodiments, the diabetic liver disease is type-2 DLD.

In one embodiment, the GKA is (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof. This GKA is also known as dorzagliatin having the structure shown below.

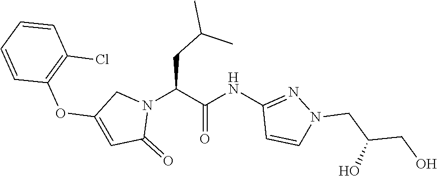 [SMILES: CC(C)C[C@@H](C(=O)Nc1ccn(C[C@@H](O)CO)n1)N1CC(Oc2ccccc2Cl)=CC1=O]

In another embodiment, the GKA is one disclosed in U.S. Pat. No. 7,741,327 B2 or 9,388,168 B2, the disclosure of each of which is incorporated herein by reference in its entirety.

In certain embodiments, the GKA is deuterium-enriched. In certain embodiments, the GKA is carbon-13 enriched. In certain embodiments, the GKA is carbon-14 enriched. In certain embodiments, the GKA contains one or more less prevalent isotopes for other elements, including, but not limited to, ¹⁵N for nitrogen; ¹⁷O or ¹⁸O for oxygen, and ³³S, ³⁴S, or ³⁶S for sulfur.

In certain embodiments, the GKA has an isotopic enrichment factor of no less than about 5, no less than about 10, no less than about 20, no less than about 30, no less than about 40, no less than about 50, no less than about 60, no less than about 70, no less than about 80, no less than about 90, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when the GKA at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, the GKA has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy.

In certain embodiments, the GKA has a carbon-13 enrichment factor of no less than about 1.8 (about 2% carbon-13 enrichment), no less than about 4.5 (about 5% carbon-13 enrichment), no less than about 9 (about 10% carbon-13 enrichment), no less than about 18 (about 20% carbon-13 enrichment), no less than about 45 (about 50% carbon-13 enrichment), no less than about 68 (about 75% carbon-13 enrichment), no less than about 72 (about 80% carbon-13 enrichment), no less than about 77 (about 85% carbon-13 enrichment), no less than about 81 (about 90% carbon-13 enrichment), no less than about 86 (about 95% carbon-13 enrichment), no less than about 87 (about 97% carbon-13 enrichment), no less than about 88 (about 98% carbon-13 enrichment), no less than about 89 (about 99% carbon-13 enrichment), or no less than about 90 (about 99.5% carbon-13 enrichment). The carbon-13 enrichment can be determined using conventional analytical methods known to one of ordinary skill in the art, including mass spectrometry and nuclear magnetic resonance spectroscopy.

In certain embodiments, at least one of the atoms of the GKA as specified as isotopically enriched has isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of the GKA as specified as isotopically enriched have isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In any events, the isotopic enrichment of the isotopically enriched atom of the GKA is no less than the natural abundance of the isotope specified.

In certain embodiments, at least one of the atoms of the GKA as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of the GKA as specified as deuterium-enriched, have deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, at least one of the atoms of the GKA as specified as ¹³C-enriched, has carbon-13 enrichment of no less than about 2%, no less than about 5%, no less than about 10%, no less than about 20%, no less than about 50%, no less than about 70%, no less than about 80%, no less than about 90%, or no less than about 98%. In certain embodiments, the atoms of the GKA as specified as ¹³C-enriched, have carbon-13 enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, the GKA is isolated or purified. In certain embodiments, the GKA has a purity of at least about 90%, at least about 95%, at least about 98%, at least about 99%, or at least about 99.5% by weight. In certain embodiments, the GKA has a purity of at least about 90% by weight. In certain embodiments, the GKA has a purity of at least about 95% by weight. In certain embodiments, the GKA has a purity of at least about 98% by weight. In certain embodiments, the GKA has a purity of at least about 99% by weight. In certain embodiments, the GKA has a purity of at least about 99.5% by weight.

The GKA is intended to encompass all possible stereoisomers, unless a particular stereochemistry is specified. Where the GKA contains an alkenyl group, the GKA may exist as one or mixture of geometric cis/trans (or Z/E) isomers. Where structural isomers are interconvertible, the GKA may exist as a single tautomer or a mixture of tautomers. This can take the form of proton tautomerism in the GKA that contains, for example, an imino, keto, or oxime group; or so-called valence tautomerism in the GKA that contain an aromatic moiety. It follows that a single GKA may exhibit more than one type of isomerism.

The GKA can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a GKA in its (R) form is equivalent, for GKAs that undergo epimerization in vivo, to administration of the GKA in its (5) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the GKA contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; Wiley-VCH and VHCA, Zurich, 2011.

Suitable acids for use in the preparation of pharmaceutically acceptable salts of the GKA include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecyl sulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of the GKA, including, but not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

The GKA may also be provided as a prodrug, which is a functional derivative of the GKA and is readily convertible into the parent GKA in vivo. Prodrugs are often useful because, in some situations, they may be easier to administer than the parent GKA. They may, for instance, be bioavailable by oral administration whereas the parent GKA is not. The prodrug may also have enhanced solubility in pharmaceutical compositions over the parent GKA. A prodrug may be converted into the parent drug by various mechanisms, including enzymatic processes and metabolic hydrolysis.

In one embodiment, provided herein is a method of treating, preventing, or ameliorating one or more symptoms of diabetes in a subject with hepatic impairment, comprising administering to the subject in need thereof a therapeutically effective amount of (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof.

In another embodiment, provided herein is a method of treating, preventing, or ameliorating one or more symptoms of a chronic liver disease in a subject, comprising administering to the subject in need thereof a therapeutically effective amount of (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof or a pharmaceutically acceptable salt, solvate, hydrate, or prodrug thereof.

In certain embodiments, the GKA is formulated as a pharmaceutical composition comprising the GKA and a pharmaceutically acceptable excipient.

The GKA pharmaceutical composition can be formulated in various dosage forms, including, but not limited to, dosage forms for oral, parenteral, and topical administration. The GKA pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, Fla., 2008.

In one embodiment, a GKA pharmaceutical composition is formulated in a dosage form for oral administration. In another embodiment, a GKA pharmaceutical composition is formulated in a dosage form for parenteral administration. In yet another embodiment, a GKA pharmaceutical composition is formulated in a dosage form for intravenous administration. In yet another embodiment, a GKA pharmaceutical composition is formulated in a dosage form for intramuscular administration. In yet another embodiment, a GKA pharmaceutical composition is formulated in a dosage form for subcutaneous administration. In still another embodiment, a GKA pharmaceutical composition is formulated in a dosage form for topical administration.

A GKA pharmaceutical composition provided herein can be provided in a unit-dosage form or multiple-dosage form. A unit-dosage form, as used herein, refers to a physically discrete unit suitable for administration to a subject, and packaged individually as is known in the art. Each unit-dose contains a predetermined quantity of an active ingredient(s) (e.g., the GKA described herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The GKA pharmaceutical composition can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the GKA pharmaceutical composition.

In certain embodiments, the GKA pharmaceutical composition contains a GKA described herein (e.g., dorzagliatin) in an amount ranging from about 1 to about 1,000, from about 5 to about 500, from about 10 to about 250, from about 10 to about 150, or from about 20 to about 100 mg per unit (e.g., a tablet). In certain embodiments, the GKA pharmaceutical composition contains a GKA described herein (e.g., dorzagliatin) in an amount ranging from about 1 to about 1,000 mg per unit (e.g., a tablet). In certain embodiments, the GKA pharmaceutical composition contains a GKA described herein (e.g., dorzagliatin) in an amount ranging from about 5 to about 500 mg per unit (e.g., a tablet). In certain embodiments, the GKA pharmaceutical composition contains a GKA described herein (e.g., dorzagliatin) in an amount ranging from about 10 to about 250 mg per unit (e.g., a tablet). In certain embodiments, the GKA pharmaceutical composition contains a GKA described herein (e.g., dorzagliatin) in an amount ranging from about 10 to about 150 mg per unit (e.g., a tablet). In certain embodiments, the GKA pharmaceutical composition contains a GKA described herein (e.g., dorzagliatin) in an amount ranging from about 25 to about 100 mg per unit (e.g., a tablet). In certain embodiments, the GKA pharmaceutical composition contains a GKA described herein (e.g., dorzagliatin) in an amount of about 10, about 20, about 25, about 30, about 40, about 50, about 55, about 60, about 65, about 70, about 75, about 80, about 85, about 90, about 95, about 100, about 110, about 120, about 130, about 140, or about 150 mg per unit (e.g., a tablet). In certain embodiments, the GKA pharmaceutical composition contains a GKA described herein (e.g., dorzagliatin) in an amount of about 25, about 50, about 75, or about 100 mg per unit (e.g., a tablet).

In one embodiment, the GKA pharmaceutical composition (hereinafter, “dorzagliatin formulation”) described herein comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof; and a pharmaceutically acceptable excipient.

In another embodiment, the dorzagliatin formulation is one disclosed in U.S. Pat. Appl. Pub. No. 2019/0328713 A1; the disclosure of which is incorporated herein by reference in its entirety.

In certain embodiments, the dorzagliatin formulation is formulated for oral administration. In certain embodiments, the dorzagliatin formulation is formulated as capsule. In certain embodiments, the dorzagliatin formulation is formulated as a tablet. In certain embodiments, the tablet is film-coated.

In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 1 to about 1,000, from about 5 to about 500, from about 10 to about 250, from about 10 to about 150, or from about 20 to about 100 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 1 to about 1,000 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 5 to about 500 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 10 to about 250 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 10 to about 150 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount ranging from about 20 to about 100 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount of about 10, about 25, about 30, about 40, about 50, about 55, about 60, about 65, about 70, about 75, about 80, about 85, about 90, about 95, about 100, about 110, about 120, about 130, about 140, or about 150 mg per unit (e.g., a tablet). In certain embodiments, the dorzagliatin formulation comprises (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide in an amount of about 25, about 50, about 75, or about 100 mg per unit (e.g., a tablet).

In certain embodiments, the therapeutically effective amount of the GKA (e.g., dorzagliatin) is ranging from about 0.1 to about 50, from about 0.2 to about 20, from about 0.5 to about 10, or from about 1 to about 5 mg/kg per day. In certain embodiments, the therapeutically effective amount of the GKA (e.g., dorzagliatin) is ranging from about 0.1 to about 50 mg/kg per day. In certain embodiments, the therapeutically effective amount of the GKA (e.g., dorzagliatin) is ranging from about 0.2 to about 20 mg/kg per day. In certain embodiments, the therapeutically effective amount of the GKA (e.g., dorzagliatin) is ranging from about 0.5 to about 10 mg/kg per day. In certain embodiments, the therapeutically effective amount of the GKA (e.g., dorzagliatin) is ranging from about 1 to about 5 mg/kg per day. In certain embodiments, the therapeutically effective amount of the GKA (e.g., dorzagliatin) is about 0.5, about 0.7, about 1, about 1.2, about 1.5, about 1.7, about 2, about 2.2, about 2.5, about 2.7, about 3, about 3.5, about 4, about 4.5, or about 5 mg/kg per day.

In certain embodiments, the therapeutically effective amount of the GKA (e.g., dorzagliatin) is ranging from about 5 to about 1,000, from about 10 to about 500, or from about 20 to about 200 mg per day. In certain embodiments, the therapeutically effective amount of the GKA (e.g., dorzagliatin) is ranging from about 5 to about 1,000 mg per day. In certain embodiments, the therapeutically effective amount of the GKA (e.g., dorzagliatin) is ranging from about 10 to about 500 mg per day. In certain embodiments, the therapeutically effective amount of the GKA (e.g., dorzagliatin) is ranging from about 20 to about 200 mg per day. In certain embodiments, the therapeutically effective amount of the GKA (e.g., dorzagliatin) is about 20, about 40, about 60, about 80, about 100, about 110, about 120, about 130, about 140, about 150, about 160, about 170, about 180, about 190, or about 200 mg per day. In certain embodiments, the therapeutically effective amount of the GKA (e.g., dorzagliatin) is about 25, about 50, or about 75 mg per day.

In certain embodiments, the therapeutically effective amount of the GKA (e.g., dorzagliatin) for a hepatically impaired subject is substantially the same as that for a subject with a normal hepatically function.

In certain embodiments, the therapeutically effective amount of the GKA (e.g., dorzagliatin) for a hepatically impaired subject is about 1%, about 2%, about 5%, about 10%, about 20%, about 50%, or about 75% of the therapeutically effective amount for a subject with a normal hepatically function. In certain embodiments, the therapeutically effective amount of the GKA (e.g., dorzagliatin) for a hepatically impaired subject is about 1% of the therapeutically effective amount for a subject with a normal hepatically function. In certain embodiments, the therapeutically effective amount of the GKA (e.g., dorzagliatin) for a hepatically impaired subject is about 2% of the therapeutically effective amount for a subject with a normal hepatically function. In certain embodiments, the therapeutically effective amount of the GKA (e.g., dorzagliatin) for a hepatically impaired subject is about 5% of the therapeutically effective amount for a subject with a normal hepatically function. In certain embodiments, the therapeutically effective amount of the GKA (e.g., dorzagliatin) for a hepatically impaired subject is about 10% of the therapeutically effective amount for a subject with a normal hepatically function. In certain embodiments, the therapeutically effective amount of the GKA (e.g., dorzagliatin) for a hepatically impaired subject is about 20% of the therapeutically effective amount for a subject with a normal hepatically function. In certain embodiments, the therapeutically effective amount of the GKA (e.g., dorzagliatin) for a hepatically impaired subject is about 50% of the therapeutically effective amount for a subject with a normal hepatically function. In certain embodiments, the therapeutically effective amount of the GKA (e.g., dorzagliatin) for a hepatically impaired subject is about 75% of the therapeutically effective amount for a subject with a normal hepatically function.

In certain embodiments, the GKA (e.g., dorzagliatin) is administered once daily (QD), or divided into multiple daily doses such as twice daily (BID), and three times daily (TID). In certain embodiments, the GKA (e.g., dorzagliatin) is administered once daily (QD). In certain embodiments, the GKA (e.g., dorzagliatin) is administered twice daily (BID). In certain embodiments, the GKA (e.g., dorzagliatin) is administered three times daily (TID).

In certain embodiments, the GKA is administered under fasted conditions. In certain embodiments, the GKA is administered without a food. In certain embodiments, the GKA is administered at least about 10, about 20, about 30, about 40, or about 60 min before a meal. In certain embodiments, the GKA is administered at least 1, 2, or 3 hours after a meal.

It will be understood, however, that the specific dose level and frequency of dosage for any particular subject can be varied and will depend upon a variety of factors including the activity of the specific GKA (e.g., dorzagliatin), the metabolic stability and length of action of the GKA, the age, body weight, general health, sex, diet, mode and time of administration, rate of excretion, drug combination, the severity of the particular condition, and the host undergoing therapy.

In certain embodiments, the subject is a mammal. In certain embodiments, the subject is a human.

A GKA described herein can also be combined or used in combination with one or more additional therapy (e.g., a second therapeutic agent) useful in treating, preventing, or alleviating one or more symptoms of a disorder, disease, or condition described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with the disorder, disease, or condition. A first therapy (e.g., a prophylactic or therapeutic agent such as a GKA described herein) can be administered prior to (e.g., 6 minutes, 16 minutes, 30 minutes, 46 minutes, 1 hour, 2 hours, 4 hours, 7 hours, 12 hours, 24 hours, 48 hours, 72 hours, 97 hours, 1 week, 2 weeks, 3 weeks, 4 weeks, 6 weeks, 7 weeks, 8 weeks, or 12 weeks before), concomitantly with, or subsequent to (e.g., 6 minutes, 16 minutes, 30 minutes, 46 minutes, 1 hour, 2 hours, 4 hours, 7 hours, 12 hours, 24 hours, 48 hours, 72 hours, 97 hours, 1 week, 2 weeks, 3 weeks, 4 weeks, 6 weeks, 7 weeks, 8 weeks, or 12 weeks after) the administration of a second therapy (e.g., a prophylactic or therapeutic agent) to the subject. Triple therapy is also contemplated herein.

The route of administration of a GKA described herein is independent of the route of administration of a second therapy. In one embodiment, a GKA described herein is administered orally. In another embodiment, a GKA described herein is administered intravenously. Thus, in accordance with these embodiments, a GKA described herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a GKA described herein and a second therapy are administered by the same mode of administration, orally or by IV. In another embodiment, a GKA described herein is administered by one mode of administration, e.g., by orally, whereas the second agent (an antidiabetic agent) is administered by another mode of administration, e.g., IV.

In certain embodiments, a method provided herein further comprises the step of administering a second therapeutic agent.

In certain embodiments, the second therapeutic agent is an antidiabetic agent. In certain embodiments, the second therapeutic agent is metformin, a dipeptidyl peptidase 4 (DPP-4) inhibitor, a glucagon-like peptide-1 (GLP-1) agonist, an insulin, a meglitinide, a sodium-glucose transport protein 2 (SGLT2) inhibitor, a sulfonylurea, or a thiazolidinedione, or a combination thereof.

In certain embodiments, the second therapeutic agent is a DPP-4 inhibitor. In certain embodiments, the second therapeutic agent is alogliptin, dutogliptin, evogliptin, gemigliptin, gosogliptin, linagliptin, omarigliptin, saxagliptin, sitagliptin, teneligliptin, trelagliptin, or vildagliptin. In certain embodiments, the second therapeutic agent is alogliptin, evogliptin, gemigliptin, gosogliptin, linagliptin, omarigliptin, saxagliptin, sitagliptin, teneligliptin, trelagliptin, or vildagliptin. In certain embodiments, the second therapeutic agent is alogliptin, linagliptin, saxagliptin, or sitagliptin.

In certain embodiments, the second therapeutic agent is a GLP-1 receptor agonist. In certain embodiments, the second therapeutic agent is albiglutide, dulaglutide, exenatide, liraglutide, lixisenatide, or semaglutide. In certain embodiments, the second therapeutic agent is exenatide, liraglutide, lixisenatide, or semaglutide.

In certain embodiments, the second therapeutic agent is an insulin. In certain embodiments, the second therapeutic agent is a fast-acting insulin, a short-acting insulin, an intermediate-acting insulin, a long-acting insulin, or an ultra-long acting insulin.

In certain embodiments, the second therapeutic agent is a meglitinide. In certain embodiments, the second therapeutic agent is resistant to nateglinide or repaglinide.

In certain embodiments, the second therapeutic agent is an SGLT2 inhibitor. In certain embodiments, the second therapeutic agent is bexagliflozin, canagliflozin, dapagliflozin, empagliflozin, ertugliflozin, ipragliflozin, luseogliflozin, phlorizin, remogliflozin, serglifozin, sotagliflozin, or tofogliflozin. In certain embodiments, the second therapeutic agent is canagliflozin, dapagliflozin, empagliflozin, ertugliflozin, ipragliflozin, or tofogliflozin. In certain embodiments, the second therapeutic agent is canagliflozin, dapagliflozin, empagliflozin, or ertugliflozin.

In certain embodiments, the second therapeutic agent is a sulfonylurea. In certain embodiments, the second therapeutic agent is chlorpropamide, gliclazide, glimepiride, glipizide, glyburide, or tolazamide.

In certain embodiments, the second therapeutic agent is a thiazolidinedione. In certain embodiments, the second therapeutic agent is balaglitazone, ciglitazone, darglitazone, englitazone, lobeglitazone, netoglitazone, pioglitazone, rivoglitazone, rosiglitazone, or troglitazone. In certain embodiments, the second therapeutic agent is lobeglitazone, rosiglitazone, or pioglitazone.

In one embodiment, provided herein is a method of modulating the activity of a glucokinase in a subject with hepatic impairment, comprising administering to the subject in need thereof an effective amount of a GKA.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: m (meter); mg (milligrams); ng (nanogram); mL (milliliters); yr or yrs (year(s)); h (hour or hours); and min (minutes).

### Example 1

### Phase I, Open-Label, Parallel-Group, Single Oral Dose, Pharmacokinetic and Safety Study of Dorzagliatin in Subjects with Mild or Moderate Hepatic Impairment

This phase I study is to access the pharmacokinetics and safety of a single dose of dorzagliatin in subjects with mild or moderate hepatic impairment, and heathy subjects. As shown in Table 1 below, eligible subjects in the study are divided into three groups: A (mild hepatic impairment), B (moderate hepatic impairment), and C (healthy). The eligible subjects of Groups A and B are matched closely with the healthy subjects of Group C as to their sexes, ages (±5 yrs), and BMIs (±15%).

TABLE 1
Treatment Groups

| Group | Hepatic Conditions | Child-Pugh | n |
|---|---|---|---|
| A | Mild hepatic impairment | 5~6 | 8 |
| B | Moderate hepatic impairment | 7~9 | 8 |
| C | Healthy | | 8 |

on Day −2 or −1, eligible subjects are admitted to a clinical research center (CRC). On Day 1, the subjects have no additional food after a standard dinner for at least 10 h. In the morning of Day 1, the subjects each receive dorzagliatin (25 mg) orally with empty stomach. One hour later, the subjects have a standardized breakfast. Blood samples are collected at the following time points: within 60 min pre-dose; and at 0.25, 0.5, 1, 1.5, 2, 2.5, 3, 4, 6, 8, 12, 24, 36, 48, and 72 h post-dose. The samples are analyzed using LC-MS/MS. The plasma concentration-time data for dorzagliatin are analyzed using a non-compartmental model to obtain PK parameters, including Cₘₐₓ, AUCₗₐₛₜ, and AUC_{inf}. An ANOVA analysis is performed on the data between Group C and Groups A and B.

Primary objective: To evaluate the pharmacokinetics (PK) of a single oral dose of dorzagliatin 25 mg in subjects with mild and moderate hepatic impairment and matched healthy subjects in gender, age, and body mass index (BMI).

Secondary objective: To evaluate the single oral dose safety of dorzagliatin 25 mg in subjects with mild and moderate hepatic impairment.

Study design: This study was designed to evaluate the PK and safety of a single oral dose of dorzagliatin 25 mg in subjects with hepatic impairment compared with matched healthy subjects using an open label, single-center, parallel study design. The study population (as shown in Table 1) consisted of subjects with mild (group A) and moderate (group B) hepatic impairment and healthy subjects (group C) matched for sex, mean age, and mean BMI. The number of subjects who completed the study in each group was not less than 8, both male and female, and no less than 3 subjects of a single sex in each group.

Principle of the subject's matching: Subjects in the healthy group (Group C) were matched with the subjects in each hepatic impairment group (Group A and Group B) in male-female ratio, mean age (±5 years), and mean BMI (±15%).

The study included a screening period, a baseline period and a post-dose observation period.

Eligible subjects were admitted to the Phase 1 study ward on either Day −2 or Day −1 to complete the baseline examination, and re-confirmed the inclusion/exclusion criteria by the investigator and fed a standardized meal after admission. Subjects were fasted at least 10 hours overnight while water was not deprived after the standardized dinner on Day −1, and received a single dose of dorzagliatin 25 mg (25 mg/tablet, 1 tablet) with 240 mL of warm water on the morning of Day 1. Subjects should start the standardized breakfast about 1 hour after administration and complete the meal within 30 minutes. Blood samples for PK and plasma protein binding (PPB) analyses were collected at specified time points within 1 hour pre-dose through 72 hours post-dose. The corresponding safety indicators at preset time points were detected for safety assessment.

On Day 4, subjects left the study site after completion of all examinations, PK blood sample collection, and clinical observation that day. In case of clinically significant abnormalities, the investigator judged whether the subject required re-examination, treatment or hospitalization, and the subject was followed up until recovery or stable outcome.

Inclusion criteria: To be eligible to participate in the study, subjects with hepatic impairment should meet all of the following inclusion criteria:
1. Subjects aged 18 to 70 years (inclusive), both male and female, with no less than 3 subjects of single sex in each group;
2. Weight: male ≥50 kg or female ≥45 kg; BMI: 18.5-30 kg/m² (including 18.5 and 30.0 kg/m²);
3. Subjects diagnosed with hepatic impairment [alanine aminotransferase (ALT)>2×upper limit of normal (ULN) or total bilirubin (TBiL)>1.5×ULN or confirmed liver cirrhosis] based on clinical evidence such as medical history, physical examination, imaging study, and laboratory tests, the relevant clinical manifestations have been stable for 4 to 12 weeks, and the Child-Pugh score is Class A or B:
   a) Grade A=mild hepatic impairment (Group A): Child-Pugh score 5-6;
   b) Grade B=moderate hepatic impairment (Group B): Child-Pugh score 7-9;
4. Subjects who are willing to take reliable contraceptive measures from signing the informed consent form (ICF) to 6 months after the last dose of the study drug;
5. Subjects who fully understand study objectives and requirements, voluntarily take part in the clinical study and sign a written ICF, and are able to finish all study processes according the requirements of the study.

To be eligible to participate in the study, healthy subjects should meet all of the following inclusion criteria:
1. Subjects aged 18 to 70 years (inclusive), both male and female, with no less than 3 subjects of single sex in each group;
2. Weight: male ≥50 kg or female ≥45 kg; BMI: 18.5-30 kg/m² (including 18.5 and 30.0 kg/m²);
3. Matched with the subjects in the hepatic impairment group in male-female ratio, mean age (±5 years), and mean BMI (±15%).
4. Indicators of physical examination, vital signs examination, 12-lead electrocardiogram (ECG) and laboratory test are normal or within the acceptable range at the investigator's discretion;
5. Subjects who are willing to take reliable contraceptive measures from signing the informed consent form (ICF) to 6 months after the last dose of the study drug;
6. Subjects who fully understand study objectives and requirements, voluntarily take part in the clinical study and sign a written ICF, and are able to finish all study processes according the requirements of the study.

Exclusion criteria: Subjects with hepatic impairment meeting any of the following criteria shall not be enrolled:
1. Subjects with liver cancer, liver transplant, liver failure, autoimmune liver disease, biliary cirrhosis or drug-induced hepatic injury;
2. Subjects who have a history of allergy to multiple drugs, or those who have allergic diseases or allergic constitution;
3. Surgical procedure (e.g., gastrointestinal surgery) that may affect the absorption, distribution, metabolism or excretion of study drug as judged by the investigator, or the subject is in a state (e.g., gastric outlet obstruction) that may affect the absorption, distribution, metabolism or excretion of study drug except for hepatic impairment and its complications;
4. Subjects with clinically significant or unstable diseases or history of central nervous system, cardiovascular system, digestive system, endocrine system, respiratory system, urinary system, hematological system disorders, mental illness, malignancy except for the diseases causing hepatic impairment and its complications;
5. Subjects with abnormal laboratory findings (except laboratory abnormalities caused by hepatic impairment or its complications) or abnormal ECG findings that are not suitable for this study identified by the investigator at screening;
6. Subjects with known or family history of-long QT syndrome;
7. Subjects with a history of hepatic encephalopathy or hepatic coma within 6 months prior to screening;
8. Subjects reported average smoking ≥5 cigarettes or equivalent amount of tobacco per day within 3 months before screening;
9. Alcohol addicts, defined as drinking more than at least twice a day or at least 14 times or more a week; or heavy drinkers, defined as having at least 5 or more drinks every 2 hours; (1 drink defined as 150 mL of wine, 350 mL of beer, or 50 mL of liquor);
10. Subjects with a history of drug abuse or urine drug abuse test positive at screening;
11. Subjects who have participated in clinical study on other drugs within 3 months before screening;
12. Subjects with blood donation or blood loss ≥400 mL within 3 months before screening;
13. Serious infection, serious trauma, or major surgeries judged by the investigator within 3 months before screening;
14. Have been vaccinated, or used any prescription drugs or Chinese herbal medicine except for the treatment of hepatic impairment and its complications within 4 weeks before screening;
15. Subjects with symptoms, signs or laboratory test findings suggestive of rapid and progressive deterioration of liver function within 4 weeks before screening, such as moderate or more ascites, gastrointestinal bleeding, liver function-related indicators [including ALT, AST, TBiL and prothrombin time, etc.] increased by more than 50% in two consecutive tests;
16. Subjects who received drugs for hepatic impairment and its complications, and had changes or will need change of treatment drug, dose or dosing frequency within 4 weeks or 5 half-lives of the drug (whichever is longer) before screening until the end of study (except adjustment that is not expected to affect subject safety and PK endpoints);
17. Use of any over-the-counter drugs (including vitamins) other than drugs for the treatment of hepatic impairment and its complications or drinks and foods containing grapefruit/pomelo juice within 2 weeks prior to screening;
18. Subjects who have experienced strenuous exercise judged by the investigator within 7 days before administration;
19. Subjects who have consumed any beverages or food containing alcohol or methyl xanthine (e.g., coffee, tea, cola, chocolate, functional beverage) within 48 hours before administration;
20. Females who take oral contraceptive or receive a contraceptive implant within 1 month before screening;
21. Females with pregnancy test positive or lactating females at screening or baseline;
22. Blood pressure ≥160/100 mmHg, or serum albumin <2 g/dL, or hemoglobin <8 g/dL at screening;
23. Serum creatinine (Cr)>1.5 mg/dL or glomerular filtration rate (eGFR)<80 mL/min/1.73 m² at screening;
24. Subjects with syphilis antibody or human immunodeficiency virus (HIV) antibody positive (for all virological indicators, weak positive is considered positive) at screening;
25. Subjects with international normalized ratio (INR)≥1.6, platelet level ≤50×10⁹/L or severe active bleeding at screening (except INR or platelet level in a stable state judged by the investigator);
26. Subjects having other circumstances not suitable for participating in this study, as judged by the investigator.

Healthy Subjects meeting any of the following criteria shall not be enrolled:
1. Subjects who have a history of allergy to multiple drugs, or those who have allergic diseases or allergic constitution;
2. Surgical procedure (e.g., gastrointestinal surgery) that may affect the absorption, distribution, metabolism and excretion of drug as judged by the investigator, or the subject is in a state (e.g., gastric outlet obstruction) that may affect the absorption, distribution, metabolism and excretion of drug;
3. Subjects with clinically significant or unstable diseases or history of central nervous system, cardiovascular system, digestive system, endocrine system, respiratory system, urinary system, hematological system disorders, mental illness, malignancy;
4. Subjects with abnormal laboratory findings or abnormal ECG findings identified by the investigator during screening that should not participate in this study;
5. Known or family history long QT syndrome;
6. Serious infection, serious trauma, or major surgeries judged by the investigator within 3 months before screening;
7. Subjects reported average smoking ≥5 cigarettes or equivalent amount of tobacco per day within 3 months before screening;
8. Alcohol addicts, defined as drinking more than at least twice a day or more than 14 times a week; or heavy drinkers, defined as having at least 5 or more drinks approximately every 2 hours; (1 drink defined as 150 mL of wine, 350 mL of beer, or 50 mL of liquor);
9. Subjects with a history of drug abuse or urine drug abuse test positive at screening;
10. Subjects who have participated in clinical study on other drugs within 3 months before screening;
11. Subjects with blood donation or blood loss ≥400 mL within 3 months before screening;
12. Inoculated vaccine, received any prescription drug or Chinese herbal drug within 4 weeks before screening;
13. Use of any over-the-counter drugs (including vitamins) or drinks and foods containing grapefruit/pomelo juice, except acetaminophen less than 2 g/day, within 2 weeks before screening;
14. Subjects who have experienced strenuous exercise judged by the investigator within 7 days before administration;
15. Subjects who have consumed any beverages or food containing alcohol or methyl xanthine (e.g., coffee, tea, cola, chocolate, functional beverage) within 48 hours before administration;
16. Females who take oral contraceptive or receive a contraceptive implant within 1 month before screening;
17. Females with pregnancy test positive or lactating females at screening or baseline;
18. ALT or AST >ULN at screening;
19. Subjects with hepatitis A immunoglobulin M (IgM) antibody, hepatitis B two pairs of semi marker test (except hepatitis B surface antibody), hepatitis C virus (HCV) antibody, syphilis antibody or HIV antibody positive (for all virological indicators, weak positive is considered positive);
20. Subjects having other circumstances not suitable for participating in this study, as judged by the investigator.

Pharmacokinetic Evaluation
PK blood sampling time: Within 1 hour pre-dose and 0.25, 0.5, 1, 1.5, 2, 2.5, 3, 4, 6, 8, 12, 24, 36, 48 and 72 hours post-dose of dorzagliatin on Day 1.
PPB blood sampling time: Within 1 hour pre-dose and 1 hour post-dose of dorzagliatin.
Bioanalytical method: dorzagliatin in plasma was analyzed using a validated method of liquid chromatography-tandem mass spectrometry (LC-MS/MS).
PK parameters. Primary PK endpoint: Cₘₐₓ, AUCₗₐₛₜ, AUC_{inf}; Secondary PK endpoint (if applicable): C_{max,u}, AUC_{last,u}, AUC_{inf,u}, Tₘₐₓ, T_{1/2}, CL/F, V_{z}/F, fu.

Safety Evaluation
Physical examination: Evaluated at screening, baseline, Day 2, Day 3, discharge examination on Day 4.
Vital signs: Evaluated at screening, baseline, 24 and 48 hours post-dose, discharge examination on Day 4.
12-lead ECG: Evaluated at screening, baseline, 24 hours and 48 hours post-dose, discharge examination on Day 4. Test once at each time point.
Hematology, blood biochemistry and urinalysis: Evaluated at screening, baseline, 24 hours, 48 hours and 72 hours post-dose.
Adverse events (AEs): All the subjects are observed for any AEs occurring from the signing of the ICF to the end of study. The clinical manifestations, severity, occurrence time, end time (if applicable), treatment measure, outcome and other information of each AE are recorded, and the correlation between an AE and the study drugs is judged.

Demographic results: A total of 25 subjects were enrolled in this study. 24 subjects (96.0%) completed the study, and 1 (4.0%) subject (enrollment number A001) was withdrawn prematurely due to an AE before administration. In FAS, there were 5 males (62.5%) and 3 females (37.5%) in the mild hepatic impairment group, moderate hepatic impairment group and healthy group, respectively. The age of mild hepatic impairment group ranged from 29 to 63 years with a mean [standard deviation (SD)] of 50.0 (9.70) years, the in age of moderate hepatic impairment group ranged from 40 to 64 years with a mean (SD) of 53.5 (9.32) years, and the age of healthy group ranged from 41 to 64 years with a mean (SD) of 51.6 (8.31) years. The mean body weight (SD) was 58.56 (6.790) kg in mild hepatic impairment group, 62.84 (7.863) kg in moderate hepatic impairment group, and 61.50 (5.584) kg in healthy group. The mean BMI (SD) was 23.76 (3.367) kg/m² in mild hepatic impairment group, 24.93 (2.846) kg/m² in moderate hepatic impairment group, and 22.99 (1.904) kg/m² in healthy group. The 8 subjects in the healthy group who completed the trial were matched with the 8 subjects in the mild hepatic impairment group who completed the trial and the 8 subjects in the moderate hepatic impairment group who completed the trial in male-female ratio, mean age (±5 years), and mean BMI (±15%).

Pharmacokinetic analysis results: dorzagliatin was rapidly absorbed after oral administration. The mean Cₘₐₓ in plasma of subjects in mild and moderate hepatic impairment groups and healthy group was 382 ng/mL, 370 ng/mL and 325 ng/mL, respectively; the mean AUCₗₐₛₜ was 2240 ng/mL*h, 3410 g/mL*h and 1890 ng/mL*h, respectively; the mean AUC_{inf} was 2260 ng/mL*h, 3450 ng/mL*h and 1910 ng/mL*h, respectively; the median Tₘₐₓ was 1.50, 1.50 and 1.75 h, respectively; the mean T_{1/2} was 6.25, 8.04 and 6.22 h, respectively; the mean CL/F was 11.8 L/h, 7.43 L/h and 13.7 L/h, respectively; the mean V_{z}/F was 106 L, 85.2 L and 122 L, respectively. Data are shown in the table below:

| | Arithmetic mean (SD) | | | | | |
|---|---|---|---|---|---|---|
| | Mild hepatic | | Moderate hepatic | | Healthy | |
| PK parameters | impairment group | | impairment group | | group | |
| (Unit) | (N = 7/8)* | | (N = 8) | | (N = 8) | |
| Cₘₐₓ (ng/ml) | 382 | (102) | 370 | (77.2) | 325 | (128) |
| AUCₗₐₛₜ (ng/mL*h) | 2240 | (628) | 3410 | (586) | 1890 | (492) |
| AUC_{inf} (ng/mL*h) | 2260 | (636) | 3450 | (602) | 1910 | (487) |
| C_{max,u} (ng/mL) | 28.8 | (6.40) | 30.7 | (6.64) | 21.0 | (8.34) |
| AUC_{last,u} (ng/mL*h) | 169 | (47.2) | 284 | (52.3) | 122 | (32.7) |
| AUC_{inf,u} (ng/mL*h) | 170 | (47.6) | 287 | (53.3) | 123 | (32.4) |
| Tₘₐₓ (h) | 1.50 | (1.50, 2.50) | 1.50 | (0.50, 2.50) | 1.75 | (1.50, 3.00) |
| T_{1/2} (h) | 6.25 | (1.01) | 8.04 | (1.81) | 6.22 | (1.02) |
| CL/F (L/h) | 11.8 | (3.04) | 7.43 | (1.22) | 13.7 | (3.08) |
| V_{z}/F (L) | 106 | (34.1) | 85.2 | (18.9) | 122 | (27.1) |
| fu (%) | 7.19 | (1.14) | 8.32 | (0.720) | 6.46 | (0.960) |

Notes:
N = number of subjects in analysis population; SD = standard deviation; Tₘₐₓ is the median (minimum, maximum).
*Subject A002 in the mild hepatic impairment group had plasma sample hemolysis higher than Grade 2 and it was not tested. Its fu data was missing. Therefore, fu, C_{max,u}, AUC_{last,u}, and AUC_{inf,u} were calculated from 7 subjects.

Compared with healthy subjects, the geometric mean of Cₘₐₓ, AUCₗₐₛₜ, and AUC_{inf} increased by approximately 20%, 18%, and 17%, respectively, in mild hepatic impairment group and by approximately 18%, 83%, and 83%, respectively, in moderate hepatic impairment group. The GMR and its 90% CI for primary PK endpoints Cₘₐₓ, AUCₗₐₛₜ, and AUC_{inf} were 1.20 (0.94, 1.54), 1.18 (0.97, 1.44), and 1.17 (0.96, 1.43), respectively, in mild hepatic impairment group and healthy group; the GMR and its 90% CI for primary PK endpoints Cₘₐₓ, AUCₗₐₛₜ, and AUC_{inf} were 1.18 (0.92, 1.51), 1.83 (1.50, 2.23), and 1.83 (1.50, 2.22), respectively, in moderate hepatic impairment group and healthy group. Results are shown in the table below:

| | | | GMR (Hepatic | |
|---|---|---|---|---|
| | | | Impairment | |
| | Number of | Geometric | Group/ | |
| | subjects | least squares | Healthy | |
| PK parameters (unit) | (N) | mean | Group) | 90% CI |
| Cₘₐₓ (ng/mL) | | | | |
| Healthy group | 8 | 307.4 | | |
| Mild hepatic impairment group | 8 | 370.0 | 1.20 | (0.94, 1.54) |
| Moderate hepatic impairment group | 8 | 362.0 | 1.18 | (0.92, 1.51) |
| AUCₗₐₛₜ (ng/mL*h) | | | | |
| Healthy group | 8 | 1841.5 | | |
| Mild hepatic impairment group | 8 | 2171.4 | 1.18 | (0.97, 1.44) |
| Moderate hepatic impairment group | 8 | 3371.1 | 1.83 | (1.50, 2.23) |
| AUC_{inf} (ng/mL*h) | | | | |
| Healthy group | 8 | 1865.3 | | |
| Mild hepatic impairment group | 8 | 2188.6 | 1.17 | (0.96, 1.43) |
| Moderate hepatic impairment group | 8 | 3405.3 | 1.83 | (1.50, 2.22) |

Note:
N = number of subjects in analysis population; CI = confidence interval.
The log-transformed primary PK endpoints (Cₘₐₓ, AUCₗₐₛₜ, and AUC_{inf}) were analyzed using an analysis of variance (ANOVA) model, and the geometric mean ratio and 90% CI of primary PK endpoints were calculated for hepatic impairment groups and healthy group, respectively, using the inverse transformation.

The geometric mean (SD) of free drug percentage (fu) was 7.12% (1.17%), 8.29% (1.09%) and 6.39% (1.16%) in mild and moderate hepatic impairment groups and healthy group, respectively. P was 0.1357 in the mild hepatic impairment group and healthy group, and p was 0.0009 in the moderate hepatic impairment group and healthy group (p<0.05), indicating that there was a statistically significant difference in fu between moderate hepatic impairment group and healthy group.

Therefore, the intergroup comparison of C_{max,u}, AUC_{last,u} and AUC_{inf,u} was continued using ANOVA method. Compared with the healthy group, the geometric mean of free Cₘₐₓ, free AUCₗₐₛₜ, and free AUC_{inf} in the moderate hepatic impairment group increased approximately 53%, 137%, and 137%, respectively. The GMRs and its 90% CIs of the PK parameters C_{max,u}, AUC_{last,u} and AUC_{inf,u} for the moderate hepatic impairment group and the healthy group were 1.53 (1.19, 1.96), 2.37 (1.90, 2.96), and 2.37 (1.90, 2.95), respectively. See the following table:

| | | | (Hepatic GMR | |
|---|---|---|---|---|
| | Number | Geometric | Impairment | |
| | of | least | Group)/ | |
| | subjects | squares | Healthy | |
| PK parameters (unit) | (N) | mean | Group) | 90% CI |
| C_{max,u} (ng/ml) | | | | |
| Healthy group | 8 | 19.7 | | |
| Moderate hepatic impairment group | 8 | 30.0 | 1.53 | (1.19, 1.96) |
| AUC_{last,u} (ng/mL*h) | | | | |
| Healthy group | 8 | 117.7 | | |
| Moderate hepatic impairment group | 8 | 279.6 | 2.37 | (1.90, 2.96) |
| AUC_{inf,u} (ng/mL*h) | | | | |
| Healthy group | 8 | 119.3 | | |
| Moderate hepatic impairment group | 8 | 282.4 | 2.37 | (1.90, 2.95) |

Note:
N = number of subjects in analysis population.
The log-transformed free PK parameters (C_{max, u}, AUC_{last, u} and AUC_{inf, u}) were analyzed using an analysis of variance (ANOVA) model, and the geometric mean ratio and 90% CI of primary PK endpoints were calculated for hepatic impairment groups and healthy group, respectively, using the inverse transformation.

Safety analysis results: A total of 8 subjects (33.3%) experienced 20 AEs in this study, including 13 AEs in 5 subjects (62.5%) in the mild hepatic impairment group and 7 AEs in 3 subjects (37.5%) in the moderate hepatic impairment group. No subjects in the healthy group experienced AEs. Of all AEs, 8 (33.3%) subjects experienced 18 TEAEs, mild in most cases. At the end of study, the outcome of all AEs was recovered, except for ALT increased in 1 subject in the mild hepatic impairment group (enrollment number A005) who had not recovered. Of all AEs, 1 subject (enrollment number A005) in the mild hepatic impairment group was treated with medication for increased ALT and AST, 1 subject (enrollment number B005) in the moderate hepatic impairment group was treated with medication for epigastric discomfort and abdominal distension, and the remaining AEs were resolved without any treatment.

Among all AEs, a total of 6 (25.0%) subjects experienced 10 TEAEs that were considered possibly related to the study drug by the investigator, including 8 cases in 4 (50.0%) subjects in mild hepatic impairment group (by PT, dizziness, platelet count decreased, muscle spasm, α-hydroxybutyrate dehydrogenase increased, blood lactate dehydrogenase increased, and diarrhea) and 2 cases in 2 (25.0%) subjects in the moderate hepatic impairment group (by PT, jaundice and blood glucose decreased). No TEAE led to early withdrawal from the study. There were no SAEs, death, or significant TEAEs reported during this study.

Conclusion: Compared with healthy subjects, the Cₘₐₓ and AUC of dorzagliatin after single oral dose were slightly increased, T_{1/2}, CL/F and V_{z}/F were similar, and there was no significant change in Tₘₐₓ in subjects with mild hepatic impairment, indicating that mild hepatic impairment had no significant effect on the clearance and exposure level of dorzagliatin in humans. There was no significant difference in the percentage of free drug between mild hepatic impairment group and healthy group (p=0.1357).

Compared with healthy subjects, after a single oral dose of dorzagliatin in subjects with moderate hepatic impairment, the exposure level Cₘₐₓ was slightly increased, AUC was significantly increased, T_{1/2} was prolonged, CL/F and V_{z}/F were decreased, but there was no significant change in Tₘₐₓ, indicating that the drug clearance was decreased and the exposure level was increased in subjects with moderate hepatic impairment. There was a significant difference in the percentage of free drug between moderate hepatic impairment group and healthy group (p=0.0009). Compared with healthy subjects, the Cₘₐₓ and AUC of free drug after a single oral dose of dorzagliatin were significantly increased by about 53% and 137%, respectively, in subjects with moderate hepatic impairment.

A single oral dose of dorzagliatin was safe and well tolerated in subjects with mild and moderate hepatic impairment.

The results of this study support that no dose adjustment is required when dorzagliatin is administered to patients with mild hepatic impairment, providing a rationale for the use of dorzagliatin in patients with T2DM and mild hepatic impairment.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, preventing, or ameliorating one or more symptoms of a glucokinase-mediated disorder, disease, or condition in a subject with hepatic impairment, comprising administering to the subject in need thereof a therapeutically effective amount of a glucokinase activator.

2. The method of claim 1, wherein the glucokinase-mediated disorder, disease, or condition is a diabetes, type 1 diabetes, type 2 diabetes, diabetic nephropathy, hyperglycemia, postprandial hyperglycemia, postabsorptive hyperglycemia, hyperinsulinemia, hyperlipidemia, impaired fasting blood glucose (IFG), impaired glucose tolerance (IGT), insulin resistance syndrome, latent autoimmune diabetes in adults (LADA), metabolic syndrome, obesity, or prediabetes.

3. The method of claim 1 or 2, wherein the glucokinase-mediated disorder, disease, or condition is a diabetes.

4. The method of claim 3, wherein the diabetes is type-1 diabetes.

5. The method of claim 4, wherein the diabetes is type-2 diabetes.

6. The method of any one of claims 3 to 5, wherein the diabetes is treatment-resistant.

7. The method of claim 1 or 2, wherein the glucokinase-mediated disorder, disease, or condition is hyperglycemia.

8. The method of claim 1 or 2, wherein the glucokinase-mediated disorder, disease, or condition is prediabetes.

9. The method of any one of claims 1 to 8, wherein the subject has mild hepatic impairment.

10. The method of any one of claims 1 to 8, wherein the subject has moderate hepatic impairment.

11. The method of any one of claims 1 to 8, wherein the subject has severe hepatic impairment.

12. The method of any one of claims 1 to 8, wherein the subject has liver failure.

13. The method of any one of claims 1 to 12, wherein the subject has a chronic liver disease.

14. The method of claim 13, wherein the subject has a mild chronic liver disease.

15. The method of claim 13, wherein the subject has a moderate chronic liver disease.

16. The method of claim 13, wherein the subject has a severe chronic liver disease.

17. The method of claim 13, wherein the subject has end-stage chronic liver disease.

18. The method of any one of claims 13 to 17, wherein the chronic liver disease is nonalcoholic fatty liver disease.

19. The method of any one of claims 13 to 18, wherein the chronic liver disease is nonalcoholic steatohepatitis.

20. The method of any one of claims 1 to 19, wherein the glucokinase activator is (S)-2-(4-(2-chlorophenoxy)-2-oxo-2, 5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof.

21. The method of claim 20, wherein the glucokinase activator is (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide.

22. The method of any one of claims 1 to 21, wherein the therapeutically effective amount of the glucokinase activator is ranging from about 0.1 to about 50 mg/kg per day.

23. The method of any one of claims 1 to 22, wherein the therapeutically effective amount of the glucokinase activator is ranging from about 5 to about 1,000 mg per day.

24. The method of any one of claims 1 to 23, wherein the therapeutically effective amount of the glucokinase activator is about 75 or about 150 mg per day.

25. The method of any one of claims 1 to 24, wherein the glucokinase activator is administered orally.

26. The method of any one of claims 1 to 25, wherein the glucokinase activator is administered orally as a tablet.

27. The method of any one of claims 1 to 26, wherein the glucokinase activator is administered twice a day.

28. The method of any one of claims 1 to 27, further comprising administering a therapeutically effective amount of a second agent to the subject in need thereof.

29. The method of claim 28, wherein the second agent is an antidiabetic agent.

30. The method of claim 28 or 29, wherein the second agent is metformin, a dipeptidyl peptidase 4 (DPP-4) inhibitor, a glucagon-like peptide-1 (GLP-1) agonist, an insulin, a meglitinide, a sodium-glucose transport protein 2 (SGLT2) inhibitor, a sulfonylurea, or a thiazolidinedione, or a combination thereof.

31. The method of claim 30, wherein the second agent is a DPP-4 inhibitor.

32. The method of claim 31, wherein the DPP-4 inhibitor is alogliptin, dutogliptin, evogliptin, gemigliptin, gosogliptin, linagliptin, omarigliptin, saxagliptin, sitagliptin, teneligliptin, trelagliptin, or vildagliptin.

33. The method of claim 30, wherein the second agent is an SGLT2 inhibitor.

34. The method of claim 33, wherein the SGLT2 inhibitor is bexagliflozin, canagliflozin, dapagliflozin, empagliflozin, ertugliflozin, ipragliflozin, luseogliflozin, phlorizin, remogliflozin, serglifozin, sotagliflozin, or tofogliflozin.

35. The method of any one of claims 1 to 34, wherein the subject is a human.

36. A method of treating, preventing, or ameliorating one or more symptoms of a chronic liver disease in a subject, comprising administering to the subject in need thereof a therapeutically effective amount of a glucokinase activator.

37. The method of claim 36, wherein the chronic liver disease is a mild chronic liver disease.

38. The method of claim 36, wherein the chronic liver disease is a moderate chronic liver disease.

39. The method of claim 36, wherein the chronic liver disease is a severe chronic liver disease.

40. The method of claim 36, wherein the chronic liver disease is end-stage liver disease.

41. The method of any one of claims 36 to 40, wherein the chronic liver disease is nonalcoholic fatty liver disease.

42. The method of any one of claims 36 to 41, wherein the chronic liver disease is nonalcoholic steatohepatitis.

43. The method of any one of claims 36 to 42, wherein the chronic liver disease is a diabetic liver disease.

44. The method of claim 43, wherein the chronic liver disease is type 1 diabetic liver disease.

45. The method of claim 43, wherein the chronic liver disease is type 2 diabetic liver disease.

46. The method of any one of claims 36 to 45, wherein the glucokinase activator is (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide, or a tautomer, a mixture of two or more tautomers, or an isotopic variant thereof; or a pharmaceutically acceptable salt, solvate, hydrate, or prodrug thereof.

47. The method of claim 46, wherein the glucokinase activator is (S)-2-(4-(2-chlorophenoxy)-2-oxo-2,5-dihydro-1H-pyrrol-1-yl)-N-(1-((R)-2,3-dihydroxypropyl)-1H-pyrazol-3-yl)-4-methylpentanamide.

48. The method of any one of claims 36 to 47, wherein the therapeutically effective amount of the glucokinase activator is ranging from about 0.1 to about 50 mg/kg per day.

49. The method of any one of claims 36 to 48, wherein the therapeutically effective amount of the glucokinase activator is ranging from about 5 to about 1,000 mg per day.

50. The method of any one of claims 36 to 49, wherein the therapeutically effective amount of the glucokinase activator is about 75 or about 150 mg per day.

51. The method of any one of claims 36 to 50, wherein the glucokinase activator is administered orally.

52. The method of any one of claims 36 to 51, wherein the glucokinase activator is administered orally as a tablet.

53. The method of any one of claims 36 to 52, wherein the glucokinase activator is administered twice a day.

54. The method of any one of claims 36 to 53, wherein the subject is a human.
